(19) United States
(12) Patent Application Publication
(10) Pub. No.: US 2014/0194668 A1
(43) Pub. Date: Jul. 10, 2014

(54) Portable Therapeutic Device Using Rotating Static Magnetic Fields

(71) Applicant: DEVICE THERAPEUTICS, INC., Flushing, NY (US)

(72) Inventor: Darrin Hanson, Flushing, NY (US)

(73) Assignee: DEVICE THERAPEUTICS, INC., Flushing, NY (US)

(21) Appl. No.: 13/738,486

(22) Filed: Jan. 10, 2013

(51) Int. Cl. A61N 2/00

(52) U.S. Cl. CPC A61N 2/004
USPC 600/9

(57) ABSTRACT

The present invention provides a portable therapeutic device for treatment of dyslipidemia, hyperviscosaemia, diabetic neuropathy, and peripheral artery disease, using rotating Static Magnetic Fields (rSMF). The device comprises a two part plastic housing with a handle and a designated treatment area, a DC motor with microcontroller and a cylindrically shaped magnet roller, which comprises magnets tightly fitted into a stainless steel sleeve capped with two bearing shafts protruded on each end. The magnet roller assembly rotates freely inside the housing with little clearance at assigned low frequency in line with the DC motor axis driven by magnetic torque from magnetic couplings, which include equally sized quad-pole magnet plates on each coupling base. The rSMF-based device produces an inhomogeneous surface intensity of 4000-7000 Gauss from its dual-pole or quad-pole parallel circuitry for an effective treatment regimen of the indicated disorders.

### BACKGROUND OF THE INVENTION

1. Field of the Invention

The present invention relates to a portable medical device using rotating Static Magnetic Fields (rSMF) as its energy source, and more specifically to the field of Device-based Therapies (DBT) or Device Therapeutics. The rSMF-based device is adapted to treat dyslipidemia (hypercholesterolemia and hypertriglyceridemia), and hyperviscosaemia, and can also be used to treat diabetic neuropathy and peripheral artery disease (PAD) with clinically validated design of indication-specific treatment protocols as safe and efficacious alternatives to pharmacotherapies.

2. Description of Related Art

Researchers have long noticed and advocated that a magnetic field cannot have a desired biological effect unless there is motion in the field or the magnetic field is dynamic. Many device designs have been proposed using static magnetic field rotating either axially or bi-axially or time-varying pulsed electromagnetic field, but lack of clinically efficacious treatment protocols have resulted in conflicting views towards magnetic therapy in any form, unless verifiable therapeutic effectiveness is incontrovertibly established. It is believed that the modality with the least side effects will eventually be recognized and accepted by medical professionals in the therapeutic domain, just as Magnetic Resonance Imaging (MRI) and Magnetic Resonance Angiography (MRA), which have long been cleared for diagnostic imaging utilizing strong gradient static magnetic fields with radio frequency (RF).

The nomenclature used in this field also adds to the current state of confusion. There are several names being used in publications to describe the same type of magnetic field, such as RCMF (Rotating Constant-strength Magnetic Fields), RPMF (Rotating Permanent Magnetic Fields), RSMF (Rotating Stationary Magnetic Fields), RCSMF (Rotating Constant Strength magnetic Fields) and RSMF (Alternating Static Magnetic Fields). As used herein, rSMF (rotating Static Magnetic Field) is selected as a standard acronym for the technology in hopes of eliminating the confusing usage in future academic and clinical documentation.

The following patent disclosures relate to dynamic magnetic field treatments and may be relevant to the rSMF subject matter of the present application.

U.S. Pat. No. 4,727,857 to Alois Hörl, uses a disc shape platform consisting of two magnets placed so as to rotate and generate a pulsating electromagnetic field. The patent discloses a relatively weak intensity of 1000 Gauss and use of a pulsating mode, which may not reach the threshold of potency as to trigger any evaluable therapeutic effects.

U.S. Pat. No. 5,667,469 and Chinese utility patent No. 93118017.1 to Xiao-yun Zhang et al. provides a scheme of magnet allocation hoping to avoid any magnetic flux leak and a specific range of low rpm rotation. The bed shape design is limited to apply the surface treatment area to the target anatomical sites such as the nape of the neck, and the field intensity of 6000 to 8000 Gauss is chosen arbitrarily in a one-size-fits-all manner. The device is designed mainly for musculoskeletal rehabilitation and animal testing. More importantly, the rotating frequency is set too low for a dynamic field to effectively interact with target tissue at the cellular level for positive results.

U.S. Pat. No. 5,632,720 to Chelton R. Kleitz illustrates a design of rotating magnets in a cylindrical tube as a massage wand. The application method using the wand is impractical and the high rpm (3000-5000) and weak field intensity (950-1050 Gauss) are just a few reasons that the design is not effective as a medical device.

U.S. Pat. No. 6,001,055 to James Souder depicts a portable design with a rotating mechanism and is premised on the belief that subjecting a treatment area to a magnetic flux fields consisting of primarily north-pole flux enhances the therapeutic effect of the treatment on the anatomical area. The general description does not elucidate the critical parameters needed for setting proper treatment such as recommended intensity and frequency.

U.S. Pat. Nos. 7,354,393 and 7,507,198 B2 to Vincent Ardizzone disclose two designs of a therapeutic apparatus using a bi-axial rotating mechanism for health-related applications on either humans or animals. Commercial products based on the first patent for general wellness are called Body Energizer, Magboy and PowerMag, made by Nikken, Inc. in Irvine Calif., which all come with a surface intensity of 1800 Gauss and a 500-1200 rpm biaxial rotation mechanism and no medical claims according to the product instruction manuals. The two patents failed to show mechanical configuration parameters or any specific therapeutic applications in a clinical environment.

Chinese Utility Patent No. 200710001277.6 and International Application No. PCT/CN2007/000732 to Zhong-ping Lou have elucidated a design intended for rehabilitative applications of a wide range of indications of discomfort, which are mostly musculoskeletal-related. The layered design of magnets and ferromagnet is seriously flawed, as the assembly does not have any protection measure in the event that the unit is dropped and breaks open. The shaft coupling design will sooner or later lead to high temperature in the housing which in turn will burn and even ignite a fire when the shaft axis is not properly aligned. Furthermore, it does not provide any specifics on either the unit measurement or indications with treatment protocols.

Pending Chinese Utility Patent Applications CN 200910225052.8, CN 201010510408.5 and 201210049334.9 have employed the design idea of rotating magnetic fields but all mechanical layouts are short of specifics relating to key device and treatment parameters. They are primarily intended for rehabilitative and physical therapy or general wellness applications.

### SUMMARY OF THE INVENTION

In one aspect the invention is a portable therapeutic device, comprising: a housing made of nonmetallic material with a handle and a designated treatment area; a DC motor with a programmable microcontroller; a cylindrical magnet roller assembly comprising a stainless steel sleeve enclosing a diametrically magnetized magnet or magnets, and optionally a ferromagnet in addition to the magnets; and a pair of non-contact magnetic couplings interposed between the DC motor and the magnet roller assembly; wherein said DC motor and cylindrical magnet roller assembly produce a rotating static magnetic field for treatment of hypercholesterolemia, hypertriglyceridemia, hyperviscosaemia, diabetic neuropathy and/or peripheral artery disease.

The method of treating these one or more indications typically includes applying a rotating static magnetic field rotating at a frequency in a range of 8-15 Hz with inhomogeneous surface field intensity in a range of 4000-7000 Gauss to a designated anatomical site of a human subject for an effective treatment time. For treatment of hypercholesterolemia, hypertriglyceridemia, and/or hyperviscosaemia, the designated anatomical site of the subject is the nape of the neck and the respective treatment time is about 20-30 minutes per day. For the treatment of diabetic neuropathy and/or peripheral artery disease, the designated anatomical site is the sole of the foot and/or the calf and the effective treatment time is in a range of about 30-50 minutes per day.

### BRIEF DESCRIPTION OF THE DRAWINGS

FIG. 1 is a perspective view of a preferred embodiment of the invention.

FIG. 2 is a top view of the embodiment of FIG. 1.

FIG. 3 is a perspective view of a complete assembly of the rSMF-based device of FIG. 1, with the cover removed.

FIG. 4a is a front end view of a magnetic coupling fitting.

FIG. 4b is a perspective view of a magnetic coupling fitting.

FIG. 4c is a cross-sectional view of a magnetic coupling fitting.

FIG. 5 is a perspective view of a DC motor with shaft and a magnetic coupling fitting.

FIG. 6 is a perspective view of a magnet roller assembly with two bearings and magnetic coupling fitting.

FIG. 7 is a perspective view of an embodiment having a one-part magnet assembly.

FIG. 8 is a perspective view of an embodiment having a two-part magnet assembly.

FIG. 9 is a perspective view of an embodiment having a three-part magnet assembly.

FIG. 10 is a top view of complete magnet assembly in the bottom housing.

FIG. 11 is a perspective view of complete magnet roller assembly.

FIG. 12a is a top view of magnet assembly support notches in the bottom housing.

FIG. 12b is a fastening band as bearing cover and Hex screws.

FIG. 13 is a bottom side view of a slidable lifter with thumb screws.

FIG. 14 is a side view of a fully expended lifter.

FIG. 15 illustrates device application on a preferred anatomical site (nape of the neck).

FIG. 16 illustrates device application using an adjustable chair incorporating the device.

FIG. 17 illustrates positioning the device for application on a preferred anatomical site (sole of the foot).

### DETAILED DESCRIPTION OF THE INVENTION

The present invention addresses the curative mechanism of a rSMF-based device, providing detailed information on the treatment parameters for indications of dyslipidemia, hyperviscosaemia, diabetic neuropathy and peripheral artery disease from the viewpoint of both the mechanical configuration and dosimetric parameters.

Dyslipidemia is associated with cholesterol dysregulation such as hypercholesterolemia and hypertriglyceridemia, while hyperviscosaemia and peripheral artery disease denote irregular changes of hemo-rheological values and poor microcirculation in respect to high cholesterol, abnormal erythrocyte and platelet aggregation, which may lead to plaque buildup in the arteries of the extremities. Currently cholesterol lowering and anti-platelet drugs will be prescribed to treat these disorders such as Lipitor and Plavix, which are the brand names for statins and clopidogrel bisulfate. Oftentimes doctors prefer to put patients on an OTC Aspirin regimen that serves as a blood thinner.

Although the side effects of these two drugs prove to be detrimental to hepatic and renal functions, they are still the heavily prescribed drugs with a dominant market share and over 14 billion US dollars in combined annual sales in the US alone when dealing with high cholesterol and platelet aggregation. The long term use of these drugs has created a large population of patients whose hepatic and renal functions are impaired. This group of patients still has to receive treatment for the same indications—but is advised to stop any drug regimen by their doctors due to iatrogenic or drug-induced damages to their visceral organs, and no suitable medical treatment options have yet been presented.

Diabetic neuropathies are progressive neuropathic disorders associated with diabetes. These conditions are thought to result from diabetic microvascular injury involving small blood vessels that supply nerves in addition to macrovascular conditions that can culminate in diabetic neuropathy. Vascular and neural diseases are closely related and intertwined. Blood vessels depend on normal nerve function, and nerves depend on adequate blood flow. Despite advances in the understanding of the metabolic causes of neuropathy, treatments aimed at interrupting these pathological processes have been limited. Thus, with the exception of tight glucose control, treatments are mainly for reducing or masking pain and other symptoms with opioids. Prescription drugs approved by the FDA for the treatment of diabetic peripheral neuropathy are the antidepressants Cymbalta and Duloxetinethe, anticonvulsant Pregabalin and the antineuropathic Lyrica, which cannot be considered as cures for diabetic neuropathies at all. In many cases, the risks of side effects and adverse events from these drugs outweigh their benefits. Patients with abnormal hepatic and renal functions simply cannot take these drugs. Other attempts have been made to ease symptoms through nutritional management of endothelial dysfunction by using orally administered vitamins. Diabetics are candidates to receive the benefits of rSMF-based device therapy for improving peripheral blood circulation and eliminating pain associated with diabetic neuropathy.

The rSMF-based device and treatment according to the invention are intended for all patients with the target indications. The invention may have particular utility in terms of treatment for the following specific patient groups:

a. No Option Patients (NOP):
Chronic Liver Disease (CLD)/Drug-induced Hepatopathy
Chronic Kidney Disease (CKD)/Drug-induced Nephropathy
Pregnant and Lactating Women
Discordant Drug Therapy Responses

The most recent statistics from the American Liver Foundation indicates that one out of every 10 Americans is affected by liver diseases out of which Chronic Liver Disease (CLD) and Drug-induced Hepatopathy (DIH) are the most prevalent types among NOP. According to the American Kidney Foundation, 26 million American adults have Chronic Kidney Disease (CKD) and millions of others are at increased risk. An alarming problem relates to those patients with Drug-induced Nephropathy (DIN) which has become one of the reasons doctors believe that they should stop any prescription drugs unless it is a matter of life and death.

Pregnant and lactating women are the natural group that falls into the demographic of NOP who may not be able to address their dyslipidemia and hyperviscosaemia issues with drugs. This is a particular group of candidates who should be advised not to take any drugs for the indicated problems, and yet they still need intervention.

A final category in the NOP group is patients with discordant responses to drug therapy, specifically to statin regimens. About 3-4 million (3%-4%) out of 100 million Americans who have abnormal lipid profiles and do not respond well to one or few statin drugs, in some cases, the drugs simply do not work at all.

The NOP group has created a demand for a non-drug solution or device based alternative in order to continue intervention of both irregular lipid profile and plaque formation. Even though there are several ways to treat dyslipidemia other than drugs, such as diet and exercise or nutraceutical dietary supplements such as niacin, nattokinase and CoQ10, none of these natural remedies have the desirable potency needed for an immediate and exogenous impact on correcting the lipid profile, while also improving hemorheological parameters to within medically accepted values. The considerable side effects coming with these unregulated supplements may be far greater than the asserted benefits. The efforts of introducing “friendlier” drugs like Lovaza and Niaspan, which are made from fish oil and niacin in pharmaceutical grade, do not actually benefit NOPs in any way as the side effects are also severe. The unmet need of NOP is a significant motivation behind the present invention.

b. Indication-specific Group (ISG):
Hypertensive Disorders (Hypertension)
Atrial Fibrillation (AFib)
Transient Ischemic Attack (TIA or Mini Stroke)
Obesity

ISG is another group of patients who are advised to stay on blood thinners such as Coumadin, Persantin or Plavix as anticoagulation or antiplatelet therapy for preventing heart attack or stroke. Patients with the following four indications named in this category are also the optimal recipients for the treatment, management and prevention using the rSMF-based device for their pathological conditions, for the reason that many of the patients in this group cannot take blood thinners due to known side effects and adverse events.

c. Post Event Management (PEM):
Stroke
Heart Attack
CABG (Coronary Artery Bypass Graft)
PTCA (Percutaneous Transluminal Coronary Angioplasty)

PEM is a sizable group of patients who will need to receive further treatment or management of their vascular conditions right after the occurrence of the above events. These patients were normally put on a drug regimen to prevent the events from happening again. Unfortunately most of them are senile elders with poor hepatic and renal functions in the first place and they are not supposed to incur any further impairment from the chemically based medicine at all if ever an alternative or choice of therapy is available. A rSMF-based device is the non-drug option as blood thinning choice to avoid future events. The PEM group accounts for about 4.5 million new patients every year in the U.S. alone based on statistics collected from professional organizations.

Statistics show that there has been a 24% decline in heart attacks and a 62% drop in more serious heart attacks since 2000 thanks to life style change and drug interventions. US FDA, pharmaceutical companies and many educated individuals came to realize that statins such as Lipitor, Crestor, and blood thinners such as Plavix and OTC Aspirin, can be used by healthy people with family history and other risk factors, or by healthy people with simple hypercholesterolemia as a Proactive Intervention Approach (PIA), from which a new category or target group has been created:

d. Prophylactic Regimen Group (PRG):
Primary Prevention Population (PPP)
Healthy People on Statins as a Prophylactic Measure
Healthy People on Preventive Plans without Drugs
Children

Many current statin users are healthy people who do not have heart disease but who simply have high cholesterol; nonetheless, they are still advised to stay on statin drugs to reduce their cardiovascular risks despite the concerns of side effects.

Although having healthy people using drugs as a preventive arrangement is still debatable, the argument of the risk versus the benefit will definitely be settled by the time a new treatment solution with no known side effects becomes available. Most health conscious people using dietary supplements as a non-drug approach to keep fit without knowing that improper choices may result in more harm than merit. The rSMF-based device is just the right candidate for daily preventive regimen together with proper diet and exercise, or to serve as a first line of defense when tackling the target indications.

FDA has approved statin use on children who have high cholesterol as young as two years of age. The choice of therapy may affect many aspects of health for those who are to take the drug.

e. Post-operative Management (POM):
Knee/Hip Replacement: Deep Vein Thrombosis (DVT) and Pulmonary Embolism (PE)
Mechanical heart valve replacement
Other Orthopedic Implants
General Surgery: Venous Thromboembolism (VTE)

In the hours and days following surgery, risk of developing venous thromboembolism (VTE) is fairly high, even for healthy patients. Venous thromboembolism is an umbrella term for deep vein thrombosis (strands of protein that prompt blood clotting in the legs) and pulmonary embolism (blockage of a lung artery from a clot broken away from the legs).

Post surgery patients with high risk factors such as diabetes, heart diseases and hypertensive disorders should be extremely cautious about the possible occurrence of VTE and they ought to be included in the PEM (Post Event Management) group so their need to avoid any further complications can be addressed by reviewing the choices of therapy other than pharmaceuticals.

All of the above mentioned patient groups except PRG (Prophylactic Regimen Group) will have to continue taking drugs to treat or manage the symptoms of their respective pathological conditions per doctors' orders. For example, AFib patients need to first control their heart rate and rhythm with either drug or surgical interventions such as disopyramide and acebutolol (antiarrhythmic drugs) and Pulmonary Vein Ablation or Pulmonary Vein Antrum Isolation (PVAI). rSMF-based device only serves as a non-drug alternative for all patient groups in accordance with their need of lipid panel regulation and anticoagulation or antiplatelet therapy with prescriptions for use in either clinical or non-clinical settings (i.e., in the clinic or at home).

The conventional approach for diagnosing dyslipidemia normally depends on blood test to evaluate the following biochemical parameters:

1) Total Cholesterol (TC)

2) Triglyceride (TG)

3) High Density Lipoprotein (HDL)

4) Low Density Lipoprotein (LDL)

It is a widely accepted practice that regulating or correcting these four numbers will reduce the chance of developing heart attack and stroke. But other critical factors such as hemorheological parameters and biomarkers, which are often ignored or underrated because of different opinions on referential importance, have recently earned much ground in clinical diagnostics, including:

5) Hemorheological Tests and

6) Biomarkers.

Hemorheological testing includes these major parameters: whole blood shear viscosity, hematocrit, whole blood reductive viscosity, fibrinogen and platelet aggregation rate. The information from this test will determine the risk factors of blood viscosity which is one of the crucial components in the evaluation process according to the present invention. Biomarkers represent the tests on finding the presence and changes of apollpoprotein, C-reactive protein (CRP), Activated protein C resistance (APCR) and Lp-PLA2 (PLAC) as to assess the probability of risks in developing heart disease. These two sets of categorical parameters also play irreplaceable roles in collectively monitoring the condition and development of vascular abnormalities and should be taken into account when rendering diagnosis.

rSMF-based device therapy happens to be the solution to fulfill the unmet need for NOP, ISG, PEM, PRG, and POM or other patients who prefer not to take drugs, for the treatment and prevention of dyslipidemia, hyperviscosaemia, diabetic neuropathy and peripheral artery disease.

Although described herein specifically as a non-drug solution to treat dyslipidemia, hypeviscosaemia, diabetic neuropathy and peripheral artery disease, other vascular-related disorders that potentially may be treatable with the rSMF device of the invention include: 1) Atherosclerotic Thrombosis; 2) Coronary Artery Inflammation; 3) Carotid Artery Inflammation; 4) Jugular Vein Inflammation; 5) Chylemia; and 6) Hypertensive Disorder.

Certain exclusions as to treatment groups inevitably apply. rSMF-based device is contraindicated in:

1) patients with Hemophilia or Coagulopathy and other bleeding disorders;

2) patients with Fibrinolytic Syndrome;

3) patients with Myasthenia Gravis;

4) patients with confirmed carcinoma;

5) patients with implanted metal medical devices such as cardiac pacemakers, implantable cardiac defibrillators (ICD); insulin pumps; hepatic artery infusion pumps; vagus nerve stimulator, cochlear implant, etc.;

6) Patients with history of allergic reactions to SMF and rSMF;

7) Patients with Congenital Factor XIII Deficiency; and

8) rSMF-based device is also contraindicated 60 days before and 30 days after major surgery.

### Treatment Parameters

Ten treatment parameters are generally recognized as qualifying determinants or prerequisites for an evaluable magnetic field treatment:

1. Magnet materials

2. Magnet dimensions

3. Pole configuration

4. Measured field strength

5. Frequency of application

6. Duration of application

7. Site of application

8. Magnet support device

9. Target tissue

10. Distance from magnet surface

The treatment parameters according to the present invention have been developed specifically for the above indications in line with the device mechanical configuration using rSMF. These treatment parameters can be categorized as mechanical parameters and dosimetric parameters.

Mechanical parameters include: 1) the size of NdFeB magnets used; 2) dual pole or quad-pole magnet allocation scheme with or without shunt; 3) whether diametric magnetization is used, and 4) selection of anatomical sites and target tissue, (for example (a) cervical dorsa (carotid artery and jugular vein); (b) carpi (radial artery); (c) femoribus internus (femoral artery); (d) foot sole and/or the calf (micro vessels, nerve ending receptors and arteries in the lower leg); and (e) direct skin contact in designated anatomical sites).

The dosimetric parameters relate to therapeutic thresholds critical for the best clinical effects for target indications as follows: Inhomogeneous surface field intensity of 4000-7000 Gauss; Rotating speed of 8-15 Hz; Duration of application of 20-50 Min/Day; and Frequency of application: Consecutive 6-15 days/course/month.

Further, four safety features have been specifically designed as an integral part of the device: 1) magnetic couplings between the magnet roller and the DC motor; 2) stainless steel sleeve; 3) Safety handle; and 4) Ferromagnet-shielded carrying case.

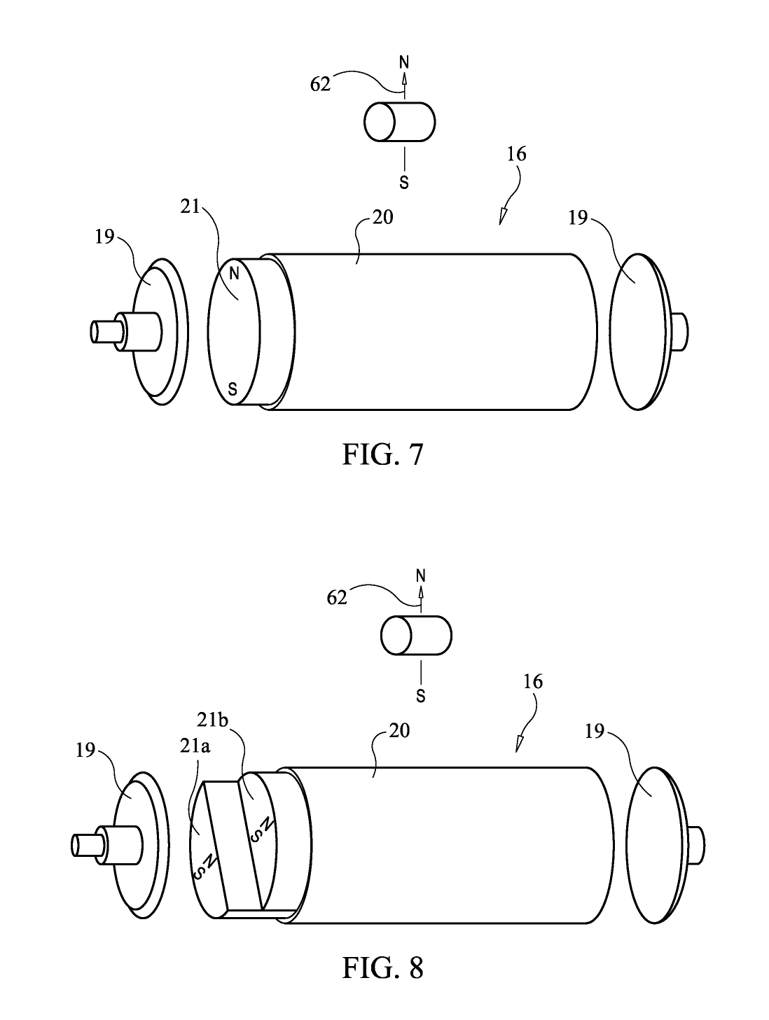

The rSMF-based portable therapeutic device consists of a two part plastic housing with a handle and a designated treatment area, a DC motor with programmable microcontroller, and a cylindrical magnet roller. The specifically sized magnet roller can be configured with either one piece of solid NdFeB (Neodymium Iron Boron) cylinder or 2 pieces of symmetrical and axially stacked NdFeB semi cylindrical magnets tightly wrapped in a stainless steel sleeve. The semi cylindrical magnet assembly can also be formed by adding a layer of ferromagnet as a shunt between the two semi cylinder magnets. The magnet roller rotates freely inside the housing at a speed of 8 to 15 Hz in line with the DC motor axis driven by magnetic torque from pair of coaxial magnetic coupling fittings on each of the DC motor and magnet roller shafts. The above components are assembled with two bearings secured in their respective slot on plastic support notches which are part of the housing base, and fastened by two semicircular bearing covers with hex screws. The magnet or plurality of magnets used in the magnet roller are diametrically magnetized, meaning that the north and south poles are oriented radially with respect to the cylinder, in a direction perpendicular to the axis of the magnet roller. The direction of diametrical magnetization is shown in FIG. 7, for example.

The housing can be made from any nonmetallic material, including plastic, fiberglass and wood, preferably ABS with soft touch design on the treatment area. One or two handles have to be included in the design for safety consideration and convenience. A slidable lifter assembly with notches is attached by two thumb screws at the back of the device to provide height adjustment for comfort and correct positioning of anatomical site.

In one preferred embodiment, one solid block of NdFeB magnet is machined to fit tightly into a circular stainless steel sleeve hermetically capped with two bearing shafts protruded on each end to make it a one part magnet assembly. In alternate embodiments, two solid, specifically sized semi cylindrical NdFeB magnets are fixed together with the opposite pole facing each other to make it a two part assembly, or alternatively, are glued onto each longitudinal side of a center layer of a ferromagnet of the same length, preferably iron, with opposite pole attracted to each side of the center layer as a three part assembly. The magnet in the magnet roller assembly is sized at 50 mm in diameter and 100 mm in length in the stainless steel sleeve which is 120 mm in length and 0.3-0.5 mm in thickness regardless of the selection of magnet roller assembly configurations. The caps with shafts protruded at the outward end are measured at 5 mm in thickness respectively. The dimension of the magnets in the magnet roller assembly can also be adjusted from 50 mm×100 mm to 60 mm×140 mm if the 3 part scheme is chosen and the treatment parameters have to be modified accordingly for the target indications. The complete assembly of diametrically magnetized magnet roller assembly, which imparts an inhomogeneous surface field intensity of 4000-7000 Gauss, rotates freely inside the housing with little clearance at assigned low frequency of 8-15 Hz in line with the DC motor axis driven by magnetic torque from magnetic couplings.

The conventional splicing of two horizontal shafts using regular coupling has many disadvantages. Different torque forces from rotation of both DC motor and magnet roller with strong rotating magnetic fields may cause distortion and misalignment that lead to excessive heat. As a result, high temperature in the housing may possibly damage or even burn the plastic enclosure. According to the invention, magnetic couplings made of stainless steel consist of two opposing round fittings are provided on each end of the shafts. A circular disc (30 mm in diameter and 3 mm in thickness), formed by four quarter circle NdFeB nickel plated magnets with a surface flux intensity of 4000-5000 Gauss, is glued to the plain side of the fitting having the same diameter. Then the magnetic coupling fittings are fixed on the shafts fastened with set screws. The torque applied to one disc is transferred through an air gap (5 mm) to the other disc. Because of its simple flat design, one can have angular misalignment of up to 3° or parallel misalignment of up to ¼″ and still transmit nearly full rotational torque. The no contact, no wear rotation mechanism is incorporated to extend the life of the device and to address potential safety concerns. More importantly, the magnetic couplings solve the heat problem by turbulent flow and spare the need for an additional cooling circuit in the microcontroller.

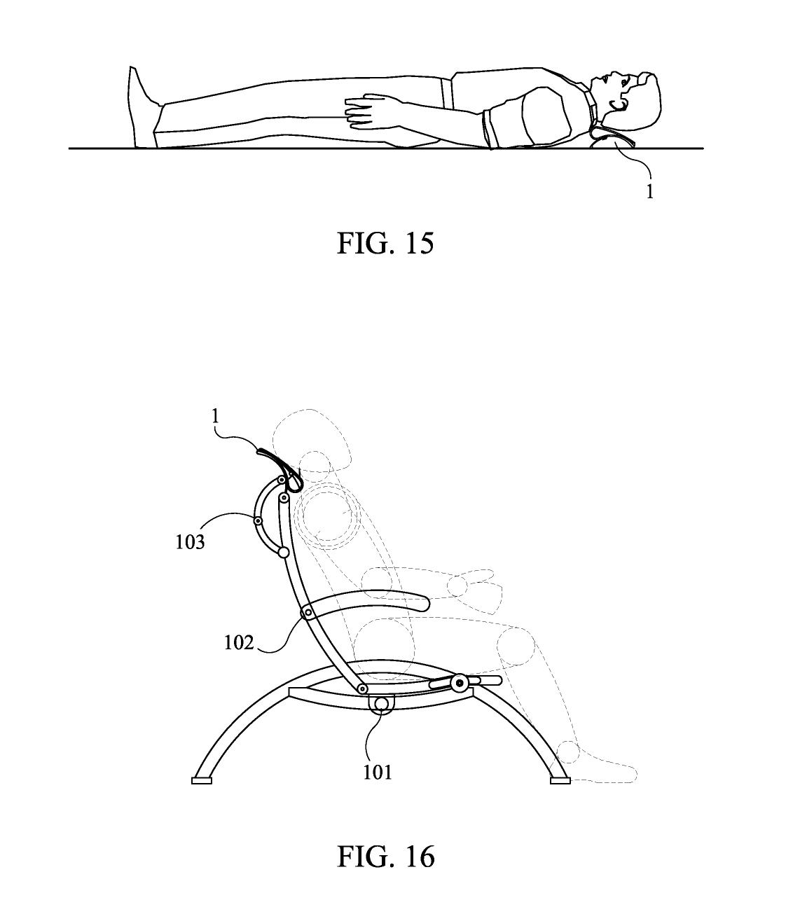

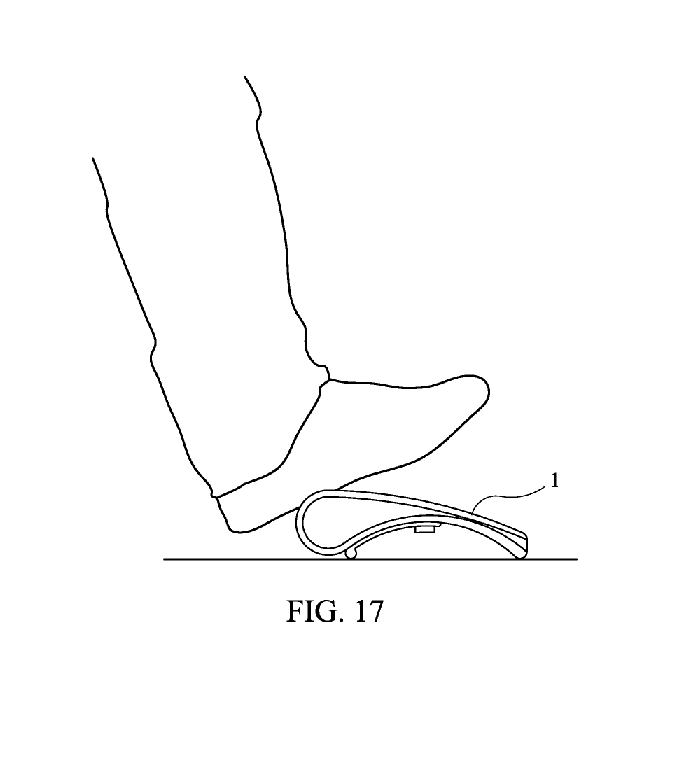

The portable rSMF-based device is designed as a DBT (Device-based Therapy) to treat and prevent dyslipidemia, hyperviscosaemia such as platelet aggregation, diabetic neuropathy and peripheral artery disease. The device must be used in direct contact with anatomical sites which specifically refer to preferably cervical dorsum (nape) as well as carpi to target the first two of the above indications, and foot sole and calf to treat the latter two. rSMF delivers dynamic magnetic force to nearby carotid artery and jugular vein when said device is used on cervical dorsum, while rSMF works on radial artery when carpi are put on the treatment area. The same principle applies to other anatomical site such as femoribus internus, foot sole and calf for said device either placed between inner thighs or under to receive extravascular rSMF exposure targeting femoral artery, micro vessels and nerve ending receptors, and arteries in the lower leg. FIG. 15 depicts an example of positioning the device under the nape of the subject's neck while the subject is in the prone position. FIG. 16 depicts a chair which may be used to position the device at an appropriate anatomical site such as the nape of the neck. FIG. 17 illustrates foot sole as another designated anatomical site to treat diabetic neuropathy.

The treatment protocol for said device includes two crucial parameters which are both time sensitive and dosage-dependent due to rSMF's cumulative and hysteresis effects. The duration of application is set at 15-50 minutes per day and the frequency of application is defined as a course of consecutive 6 to 15 days depending on specific indication. The start of a new course of treatment is based on the progress report according to scheduled blood work. These instructions have to be strictly followed in order to achieve optimal curative results, otherwise the exacerbating effect or overdose shows negative impact on target indications with elevated numbers in lipid panel when the dosimetric thresholds are exceeded.

Since NdFeB magnets used in said device are considered very strong, highly corrosive and brittle in nature, the magnet or array of magnets and ferromagnet inside said device are prone to be at risk of damage from dropping on the ground causing the magnet or stacked magnets to break loose. The stainless steel sleeve enwraps not only the magnet roller to protect it from projectile or missile effect in case of a fall but also to enhance rSMF when flux travels through the thin layer of stainless steel sleeve.

The strong static magnetic field from the device attracts any object with magnetic properties, and the danger of injuring the user's hand is very real. The design of a handle or handles is not only an added benefit for easy transport but also intended primarily as a safety feature, so a user will always carry or move said device by the handle or handles without holding the treatment area.

As a part of the treatment protocol, it is always advised that said device should be put back into its carrying case right after each daily treatment. The carrying case can be made of aluminum or plastic and is ferromagnet-shielded to protect both said device and anything is susceptible to strong magnetic fields such as credit cards, electronics and any material of ferromagnetic nature.

The design consideration for built-in safety features is equally important as that for the treatment parameters. rSMF attributes are the key to melding the therapeutic effects and safety features as one complete clinical protocol with dosimetric adequacy which can only be achieved via the use of said rSMF-based device for indication-specific applications. The detailed description will be delineated in the following sections describing the figures, where like reference characters are understood to refer to like features, elements and structure.

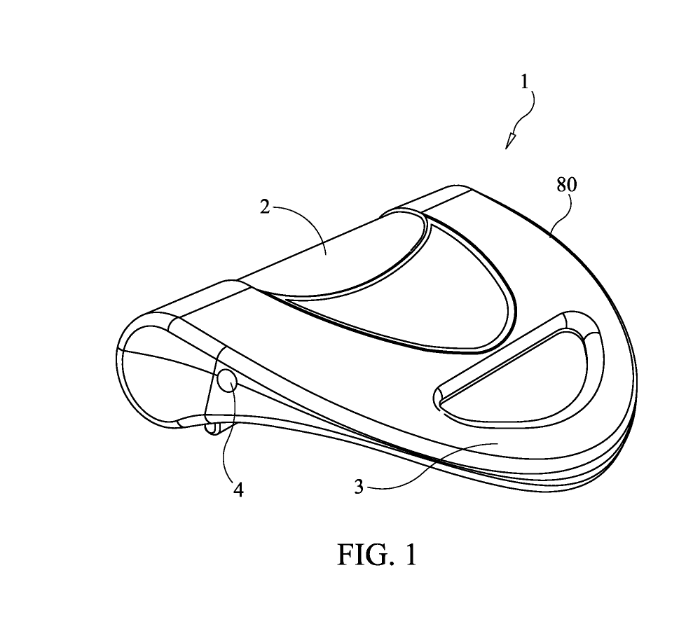

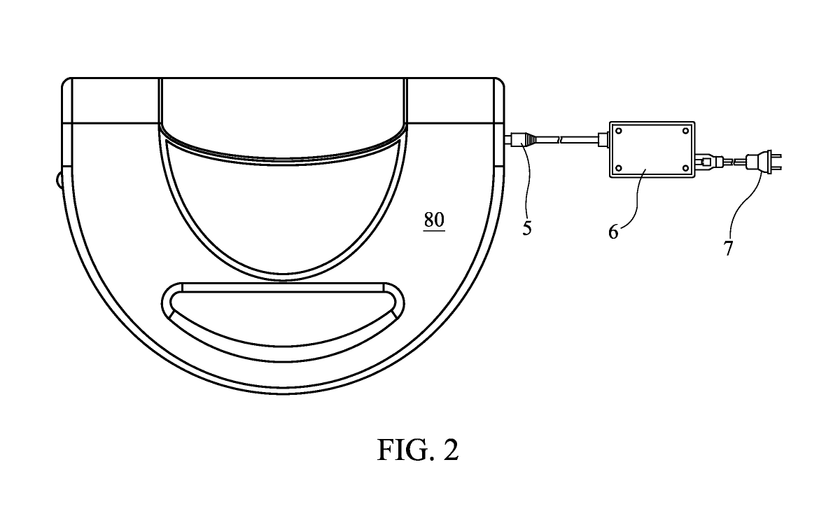

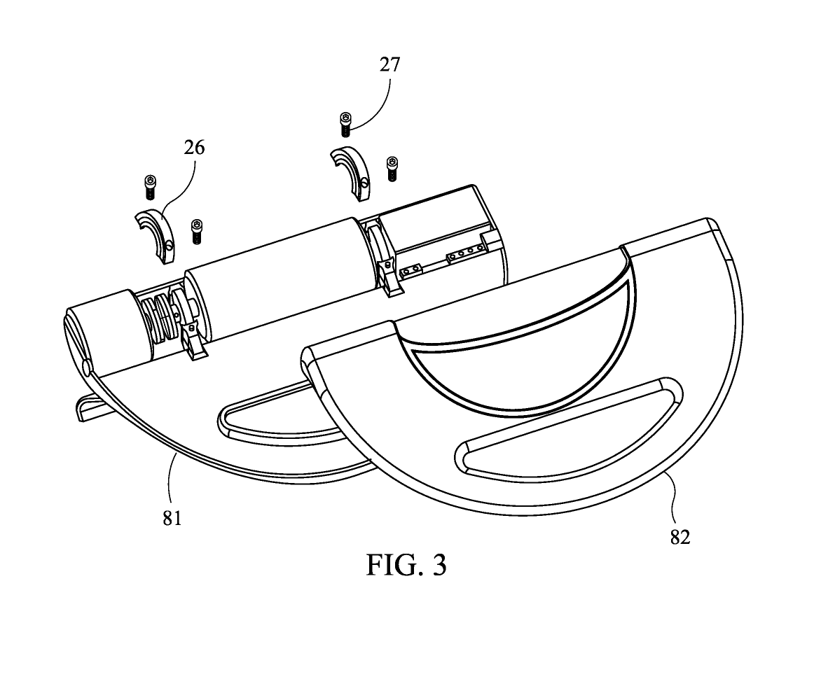

In the embodiment of FIG. 1, device 1 comprises: a two part plastic housing 80 with a soft touch treatment area 2, a membrane On/Off switch 4, and a handle 3. As shown in FIG. 2, a detachable power plug 5 with adaptor 6 and a detachable power cord 7 are provided as safety and convenience features. FIG. 3 depicts a perspective view of complete rSMF-based device assembly components with top housing 82 removed from bottom housing 81.

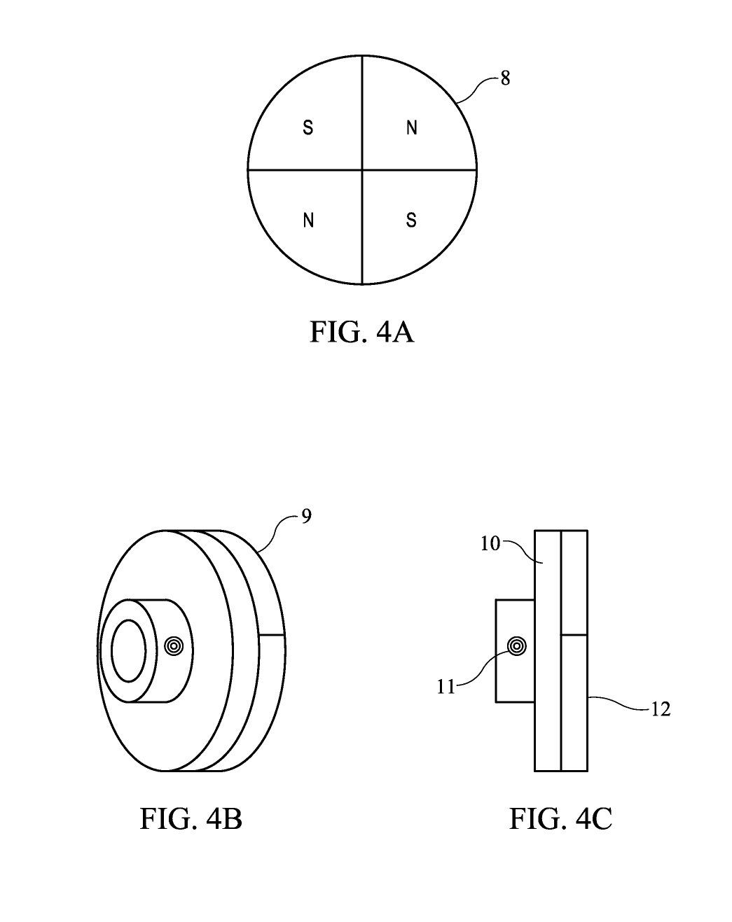

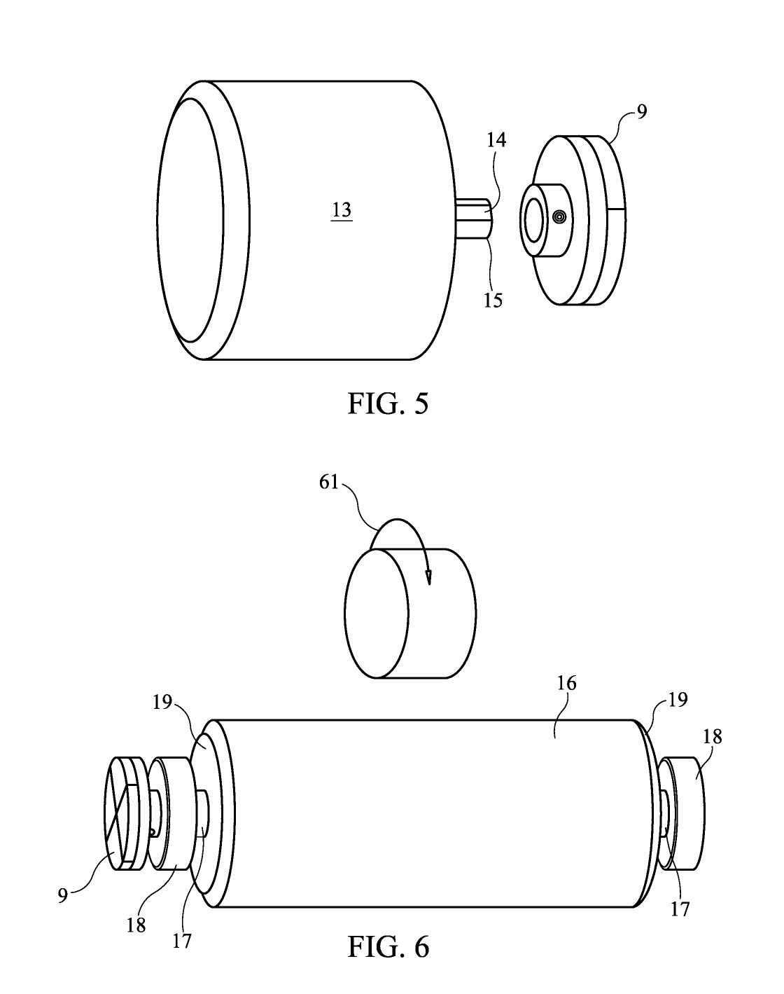

As shown in FIG. 4b, magnetic coupling fitting assembly 9 is designed to avoid misalignment of a mechanical coupling splicing, which may have the risk of damaging the housing in case of high temperature. In FIGS. 4a and 4c, the frontal end and cross-sectional side view of said magnetic coupling shows a circular shape of a magnet disc 8 (30 mm in diameter and 3 mm in thickness in the embodiment shown) and its pole orientation formed by four quarter circle NdFeB nickel plated magnets 12, which are attached to the plain side of the stainless steel fitting base 10 in same diameter by adhesive or other means. As shown in FIGS. 6, 4a and 4b, a pair of magnetic coupling fittings 9 is attached with set screws 11 on the connecting ends for final assembly. In this configuration the rSMF device can generate an equivalent field intensity of 4000-5000 Gauss from each quad-pole disc 8. The air gap is set at 5 mm between the two opposing magnetic fittings 9 based on the calculation of torque requirement to generate efficient and balanced rotation at low frequency via a DC motor 13 (FIG. 5) inside the housing.

In FIG. 5, a detent 14 machined on the end of shaft 15 from DC motor 13 is to secure the magnetic coupling fitting 9 when mounted. As shown in FIG. 6, a magnet roller assembly 16 is put together with two stainless steel or ceramic bearings 18 fixed on to both ends of the magnet roller shaft 17 via press-fit, and the two magnetic coupling fittings 9 are then inserted and secured with set screws 11 onto the shafts of both DC motor 13 and magnet roller 16. The direction of rotation of roller assembly is shown at 61 in FIG. 6, relative to the orientation of the north-south poles 62 shown in FIG. 7. The rotation of the magnet and the orientation of the poles are consistent for all of the embodiments, regardless of the configuration of the magnets.

In FIG. 7, another embodiment, having a one part magnet roller assembly 16 is disclosed in perspective view. One solid block or one solid cylindrical NdFeB magnet 21 is machined to fit tightly into a circular stainless steel sleeve 20 hermetically sealed with two bearing shafts protruded on each end of the caps 19 to make an integral magnet assembly. The single magnet configuration is primarily intended to be used for addressing dyslipidemia (hypercholesterolemia and hypertriglyceridemia) and hyperviscosaemia in view of the specific and exclusive dosimetric parameters for those treatments but it can also be used for treating diabetic neuropathy and peripheral artery disease if the housing design and treatment protocols are adapted to suit the applications to the sole of the foot and/or the calf.

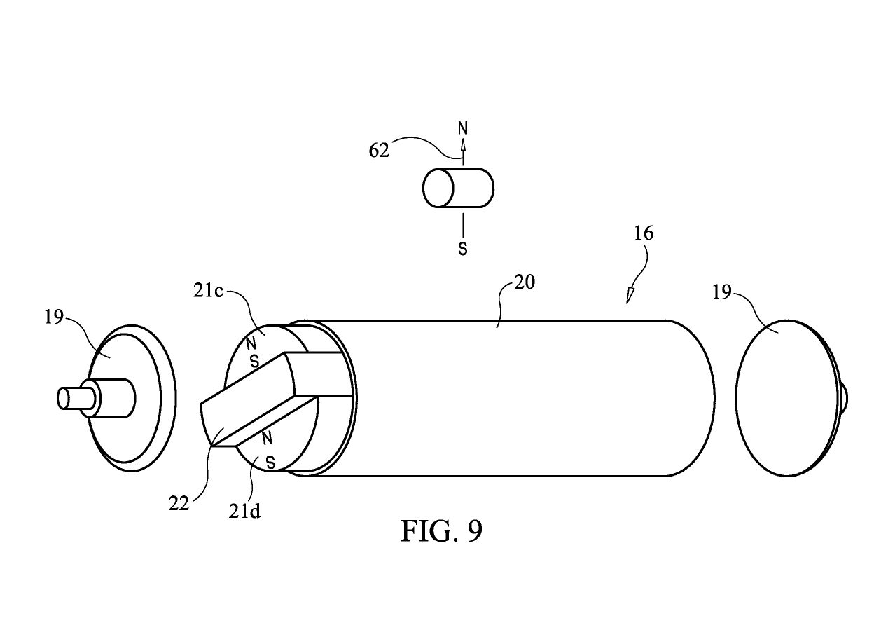

An alternate embodiments as illustrated in FIG. 8, wherein two solid, specifically sized semi cylindrical NdFeB magnets 21a, 21b are fixed together with the opposite pole facing each other to make a quad-pole, two-part assembly. As shown in FIG. 9, the two semi cylinder NdFeB magnets 21c, 21d can also be attached on either side of a center layer of a ferromagnet 22, preferably iron, of the same length, with opposite pole attracted to each side of the center layer, forming a three part assembly. In this case, both two-part and three-part magnet assemblies are machined to fit tightly into a circular stainless steel sleeve 20 for both protection and field flux enhancement. These embodiments can be applied to all treatable indications (having modified treatment and dosimetric parameters according to each specific indication of vascular pathological conditions). Both embodiments of magnet roller 16 assembly configurations can be used interchangeably in various housing designs for treating the targeted indications as long as the dosimetric values remain adequate for specific applications.

The design of NdFeB magnets 21c, 21d and ferromagnet 22 arrayed in the embodiment of FIG. 9 is a quad-pole parallel circuitry using magnet placement with ferromagnet 22 as a shunt, which imparts an inhomogeneous surface field intensity of 4000-7000 Gauss. The three part magnet roller 16 assembly works equally well as to therapeutic effects on the target indications except that it is more efficient in dealing with deeper tissues as a result of rerouted upward flux orientation as a result of ferromagnet 22.

Clinical research from both animal and human trials for the development of rSMF-based device 1 has concluded that a minimum range of 1000-1500 Gauss of field intensity is needed to reach the target cells for triggering biochemical, metabolic activities and modulating effects. The selection of magnet arrangement for the magnet roller 16 assembly is not only a matter of preference but also cost/effect consideration depending on actual clinical applications.

The magnet in the magnet roller assembly 16 in the embodiment depicted is sized at 50 mm in diameter and 100 mm in length in the stainless steel sleeve 20 which is 120 mm in length and 0.3-0.5 mm in thickness regardless of the selection of magnet roller 16 assembly configurations. The caps 19 with shafts protruded at outward end are measured at 5 mm in thickness respectively. The dimension of the magnet roller 16 can be adjusted from 50 mm×100 mm to 60 mm×140 mm if the 3 part scheme is chosen and the treatment parameters have to be modified accordingly for the target indications.

The complete diametrically magnetized magnet roller 16 assembly rotates freely inside the housing 1 with little clearance at assigned low frequency in line with the DC motor 13 axis driven by magnetic torque from magnetic couplings 9.

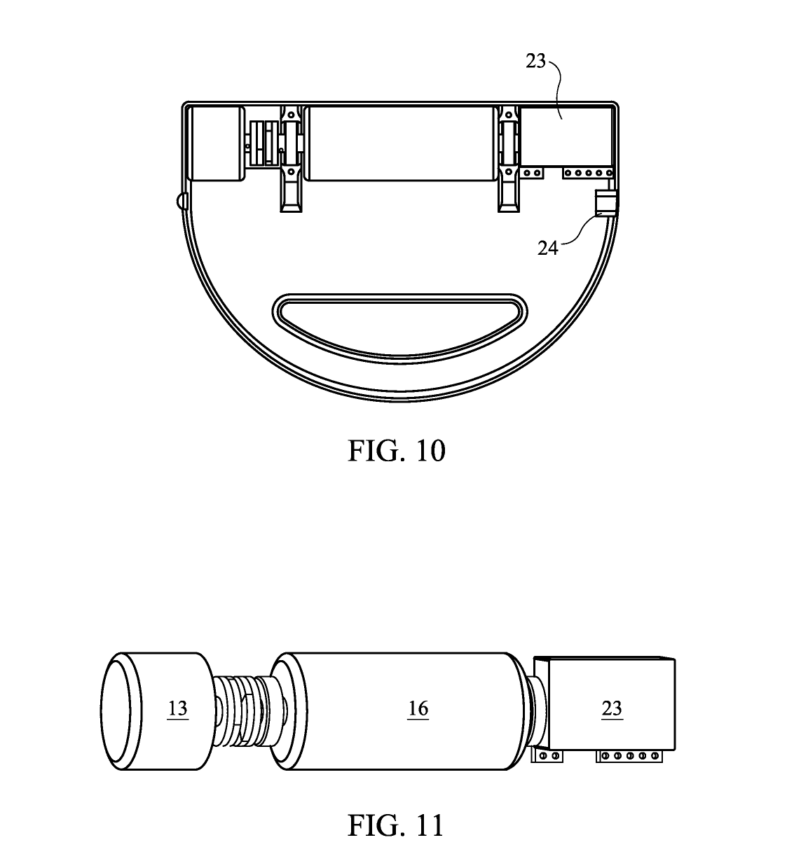

FIG. 10 shows a top view of a complete magnet roller 16 assembly with microcontroller 23 in the bottom housing, with a detachable power plug socket 24 as part of overall safety design. The microcontroller 23 box contains specially designed printed circuit board (PCB) to regulate velocity for optimal frequency. An exploded view of complete magnet roller 16 assembly in FIG. 11 illustrates the details of the components aligned for explanatory purpose.

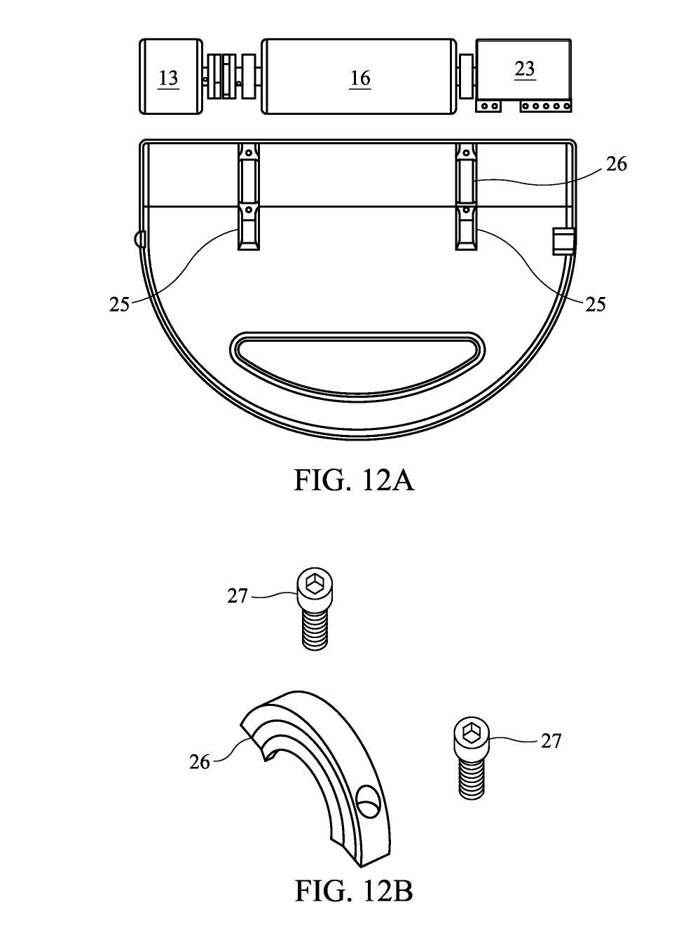

FIG. 12a displays a top view of bearing support notches 25 where the complete magnet roller 16 assembly is positioned in the bottom housing while FIG. 12b shows a perspective view of semicircular fastening band 26 as bearing cover and hex screws which are used to secure the whole magnet roller 16 assembly in place during the manufacturing process.

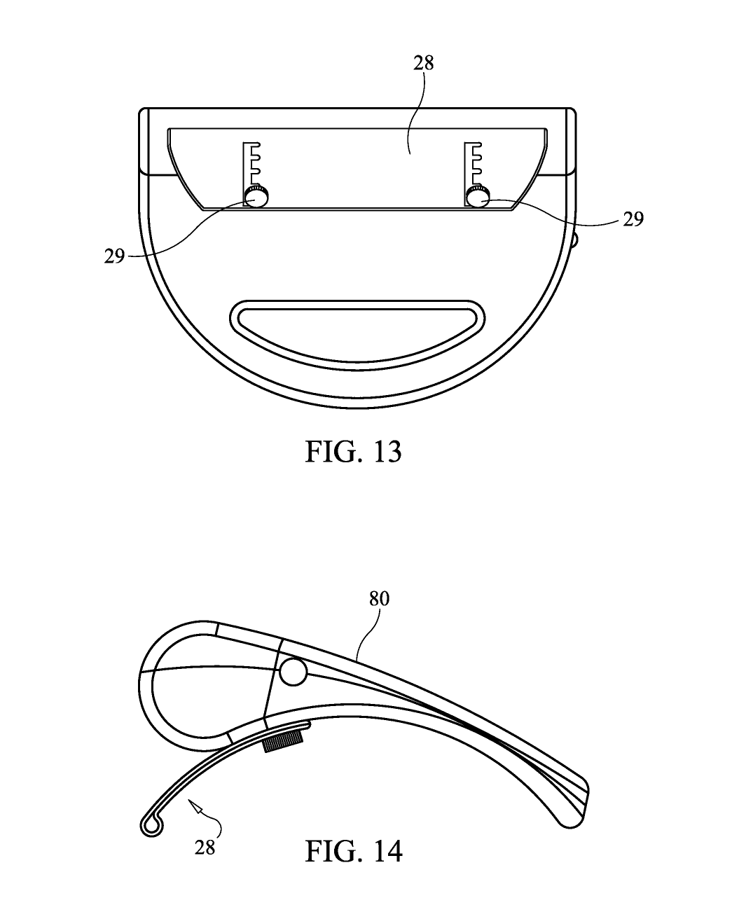

As shown in FIG. 13, a bottom side view of a slidable lifter assembly 28 with thumb screws 29 is one option for height adjustment for users' comfort and correct positioning of the anatomical treatment site. The scaled notches on the lifter assembly 28 board can be used for locking the thumb screws in place when the correct height is set. FIG. 14 illustrates a side view of a fully expended lifter 28 assembly.

FIG. 15, FIG. 16 and FIG. 17 are the illustrations of preferable anatomical sites of treatment and patient's positions associated with clinical protocol and its dosimetric parameters. In FIG. 15 the rSMF-based device 1 is placed under the cervical dorsum or nape of a patient while the patient is prone to receive antidyslipidemic (hypercholesterolemia and hypertriglyceridemia), anticoagulative or antiplatelet (hyperviscosaemia) treatments by targeting nearby carotid artery and jugular vein. Other alternate anatomical sites such as carpi or wrists, femoribus internus or inner thighs can also be used with rSMF-based device 1. Each of the alternate treatment sites corresponds to the specific target organs as carpi to radial artery, femoribus internus to femoral artery, foot sole to micro vessels and nerve ending receptors, and calf to arteries in the lower leg. FIG. 16 depicts the incorporation of a device 1 on a chair with various adjustable points 101, 102, 103. The selection of treatment sites will result in the changes in device enclosure design and dosimetric parameters to accommodate the variations according to rSMF therapeutic specificities. FIG. 17 is a detail of the device 1 applied to the sole of the foot.

The above anatomical sites have been selected as a vital component of the clinical protocol which can influence the clinical outcome very easily. Thus, each ergonomic design of said device is integrated with matching treatment and dosimetric parameters which have to be executed exactly in order to live up to its claims.

As the integral part of rSMF-based device 1 design, Ferromagnet shielded case should be used to stow away the device in both clinical and non-clinical settings when treatment sessions are done for safety reasons. In no circumstances, rSMF-based device in transport must always be stored in the shielded case, as the extremely strong pulling force of magnetic fields will surely incur serious damages or bodily injuries to the surroundings and people nearby when any larger objects of ferromagnetic nature are present.

### Example 1

A male subject, age 48, diabetic, in need of treatment for dyslipidemia, characterized by a triglyceride level of 420 mg/dL, was laid down horizontally with the nape of the neck supported by the device substantially as shown in FIG. 15. A rSMF was generated by a device substantially according to the above example, having a single NdFeB magnet rotating at a frequency of 10 Hz, having surface inhomogeneous field intensity of approximately 6000 Gauss applied with the device. The treatment was applied for twenty five minutes each day for 15 consecutive days. After each such 15-day treatment period, the subject observed a rest period of at least 20 days. The treatment was continued based on this treatment protocol within a 14 month trial period, during which period the patient's regimen of triglyceride lowering drugs was suspended. The patient continued to take Prandimet for diabetic indications. A reduction in triglyceride to 137 mg/dL was observed at the end of the trial period.

### Example 2

A female subject, age 42, in need of treatment for dyslipidemia, characterized by a total cholesterol level of 282 mg/dL was laid down horizontally with the nape of the neck supported by the device substantially as shown in FIG. 15. A rSMF is generated by a device substantially according to the above example, having a single NdFeB magnet rotating at a frequency of 10 Hz, having surface inhomogeneous field intensity of approximately 6000 Gauss applied with the device. The treatment was applied for twenty minutes each day for 6 consecutive days. After each such 6-day treatment period, the patient observed a rest period of at least 20 days. The treatment was continued based on this treatment protocol within a 14 month trial period, during which period the patient's regimen of cholesterol lowering drugs was suspended. A reduction in total cholesterol to 153 mg/dL was observed at the end of the trial period.

### Example 3

A male subject, age 58, with a three year history of confirmed diabetic peripheral neuropathy, characterized by symptoms of a tingling or burning feeling accompanies by pain and numbness. He stepped on the treatment area with one foot at a time where the sole will receive rSMF exposure as shown in FIG. 17. A rSMF is generated by a device substantially according to the above example, having a single NdFeB magnet rotating at a frequency of 10 Hz, having surface inhomogeneous field intensity of approximately 6000 Gauss is applied with a device. The treatment is applied for forty five minutes each day each foot for 15 consecutive days. After each such 15-day treatment period, the patient observes a rest period of at least 15 days. The treatment is continued based on this treatment protocol within an 8 month trial period, during which period the patient's regimen of antinociceptive and antidepressant drugs are suspended. The disappearance of all clinical symptoms associated with diabetic peripheral neuropathy is observed at the end of the trial period.

The aforementioned exemplary embodiments are illustrative of the core principles of the present invention. The schematic drawings and description are not meant to be construed as limiting the invention, which is defined by the following claims. Modifications, variations and equivalents of the invention may be made by the person of ordinary skill without departing from the spirit or scope of the claims.

1. A portable therapeutic device, comprising:
a housing made of nonmetallic material with a handle and a designated treatment area;
a DC motor with a programmable microcontroller;
a cylindrical, diametrically magnetized magnet roller assembly comprising a stainless steel sleeve enclosing a magnet or plurality of magnets; and
a non-contact magnetic coupling assembly interposed between the DC motor and the magnet roller assembly; wherein
said DC motor and cylindrical magnet roller assembly produce a rotating static magnetic field for treatment of hypercholesterolemia, hypertriglyceridemia, hyperviscosaemia, diabetic neuropathy and/or peripheral artery disease.

2. The portable therapeutic device according to claim 1, wherein the rotating speed of said cylindrical magnet roller assembly driven by said DC motor via magnetic couplings is programmed at 8-15 Hz.

3. The portable therapeutic device using according to claim 1, wherein the device produces an inhomogeneous surface field intensity of 4000-7000 Gauss.

4. The portable therapeutic device according to claim 1, wherein the cylindrical and diametrically magnetized magnet roller assembly comprises one solid block or one solid cylinder of NdFeB magnet machined to fit tightly into a hermetically sealed cylindrical stainless steel sleeve having two bearing shafts protruded on each end of the sleeve.

5. The portable therapeutic device according to claim 1, wherein two axially stacked semi-cylindrical NdFeB magnets are diametrically magnetized and fixed together with the opposite poles facing each other to form a quad-pole stack fitted tightly into a hermetically sealed cylindrical stainless steel sleeve and having two bearing shafts protruded on each end.

6. The portable therapeutic device according to claim 1, wherein said cylindrical magnet roller assembly is constructed with two semi-cylindrical NdFeB magnets placed axially on opposite sides of a ferromagnet as a center layer with opposite poles facing the center, forming a diametrically magnetized quad-pole layered stack machined to fit tightly into a hermetically sealed cylindrical stainless steel sleeve having two bearing shafts protruded on each end.

7. The portable therapeutic device according to claim 1, wherein the magnet roller assembly rotates freely inside the housing with little clearance in line with the DC motor axis driven by magnetic torque from a pair of magnetic couplings.

8. The portable therapeutic device according to claim 1, wherein said cylindrical magnet in the magnet roller assembly is sized at about 50 mm in diameter and about 100 mm in length in a stainless steel sleeve made of seamless bellows which is about 120 mm in length and about 0.3-0.5 mm in thickness.

9. The portable therapeutic device according to claim 1, wherein said magnetic coupling assembly comprises:
two opposing round fittings
a circular disk formed by four quarter-circle NdFeB nickel-plated magnets adhered to a side of the fitting having the same diameter;
both magnetic couplings facing each other being fixed on each shaft of the DC motor and the magnet roller assembly fastened with set screws, and
an air gap of 5 mm between the two magnetic couplings.

10. The portable therapeutic device according to claim 9, wherein said disk on each of said magnetic coupling fittings has a surface field intensity of 4000-5000 Gauss.

11. The portable therapeutic device according claim 10, where said disk on each of said coupling fittings has a dimension of about 30 mm in diameter and about 3 mm in thickness.

12. The portable therapeutic device according to claim 1, wherein the device has a slidable lifter with thumb screws for height adjustment and proper positioning of a patient's anatomical site during treatment sessions.

13. The portable therapeutic device according to claim 1, wherein the device has a carrying case with ferromagnet shielding integrated therein.

14. A method for treating dyslipidemia and/or hyperviscosaemia, comprising: applying a rotating static magnetic field to a human subject in need thereof for about 20-30 minutes per day with the device of claim 1 rotating at a frequency in a range of 8-15 Hz with an inhomogeneous field intensity in a range of 4000-7000 Gauss.

15. The method according to claim 14, wherein said human subject is selected from the group consisting of No Option Patients, Post Event Management Patients, Indication-Specific Group Patients; Preventive Regimen Group Patients and Post-operative Management Patients.

16. The method according to claim 14, wherein the initial frequency of application of the rotating static magnetic field is 6-15 consecutive days per course per month repeated on a monthly basis.

17. The method according to claim 14, wherein the device is placed under the subject's cervical dorsum to trigger extravascular exposure on nearby carotid artery and jugular vein.

18. A method for treating diabetic neuropathy and/or peripheral artery disease, comprising applying a rotating static magnetic field with the device of claim 1 rotating at a frequency in a range of 8-15 Hz with an inhomogeneous field intensity in a range of 4000-7000 Gauss to the foot sole and/or the calf of a patient in need thereof for about 30-50 minutes/day.

19. The method according to claim 18, wherein the initial frequency of application is set for 15 consecutive days per course per month repeated on a monthly basis.
